(19) United States
(12) Patent Application Publication
(10) Pub. No.: US 2022/0235317 A1
(43) Pub. Date: Jul. 28, 2022

(54) COMPOSITION FOR INHIBITING EXTENSION OF POPULATION DOUBLING TIME OF STEM CELLS, COMPRISING C-MET AGONIST ANTIBODY AS ACTIVE INGREDIENT

(71) Applicant: Xcell Therapeutics Inc., Seoul (KR)

(72) Inventor: Ui Il LEE, Seoul (KR)

(21) Appl. No.: 17/160,694

(22) Filed: Jan. 28, 2021

(51) Int. Cl. C12N 5/00; C12N 5/0775

(52) U.S. Cl. CPC C12N 5/0018; C12N 2501/998; C12N 5/0667

(57) ABSTRACT

The present disclosure relates to a composition for inhibiting the extension of a population doubling time of stem cells, comprising a c-Met agonist antibody as an active ingredient, and more particularly, to a medium additive for inhibiting the extension of a population doubling time of stem cells, comprising a c-Met agonist antibody as an active ingredient, a medium composition for culturing stem cells, comprising the medium additive, and a method for inhibiting the extension of a population doubling time.

### FIELD

The present disclosure relates to a composition for inhibiting the extension of a population doubling time of stem cells, comprising a c-Met agonist antibody as an active ingredient, and more particularly, to a medium additive for inhibiting the extension of a population doubling time of stem cells, comprising a c-Met agonist antibody as an active ingredient, a medium composition for culturing stem cells, comprising the medium additive, and a method for inhibiting the extension of a population doubling time.

### BACKGROUND

A stem cell is a cell having an ability to differentiate into two or more different cells while having a self-replication ability as an undifferentiated cell. Since stem cells can differentiate into cells of various tissues if they meet appropriate conditions in such an undifferentiated state, studies have been conducted to apply stem cells to treatments such as regeneration of damaged tissues.

In particular, mesenchymal stem cells are known to be pluripotent and involved in the regeneration, treatment, and immune responses of tissues, so that efforts have been made to isolate and culture mesenchymal stem cells from cord blood, bone marrow, and the like using these properties as described above and develop stem cells as a therapeutic agent for various diseases. However, there is a problem in that as stem cells are subcultured, aging progresses and stemness is lost. Specifically, results have been reported that when stem cells are subcultured, there are problems in that cell morphology, such as cell size and shape, is changed, the positive surface antigen expression of cells is reduced, and the proliferation and differentiation potential of cells is reduced, and the like. Therefore, in order to develop a cell therapeutic agent using adult stem cells without ethical problems, it is essential to establish a method for effectively proliferating stem cells while maintaining the stem ness of cells, but there is a limitation that the number of cells that can be obtained from one tissue is limited because the proliferation rate of adult stem cells is low and the adult stem cells are easily aged.

As a method for enhancing the efficiency of stem cells by solving these problems, a genetic engineering method using a viral vector or a method of overexpressing a specific protein has been proposed (Korean patent, KR10-1101835), but since the method has limited clinical application due to safety issues, its clinical applicability for the treatment of a specific disease has been verified through the first generation stem cell studies, but the efficiency is low, the effect mechanism has not been sufficiently elucidated, and the issue of securing stability has not been solved.

Meanwhile, c-Met is a representative receptor tyrosine kinase (RTK) present on the cell surface, is also called a hepatocyte growth factor receptor (HGFR), and binds to a hepatocyte growth factor/scattering factor (HGF/SF) that is a ligand thereof to promote the growth of cells by promoting intracellular signaling, and is overexpressed in many types of cancer cells, and thus is also widely involved in cancer development, cancer metastasis, cancer cell migration, cancer cell infiltration, and angiogenesis. Further, as the name of the ligand implies, c-Met signaling through HGF/SF is a representative early stage protein of cancer metastasis that weakens cell-cell contact in almost all types of epithelial tumors to cause scattering. In particular, it is well known that because there are hypoxia response elements upstream of the c-Met gene, the expression of that gene is increased in anoxic conditions. In addition, c-Met and its ligand HGF have been leading candidates for targeted cancer therapy because c-Met contributes to various stages of cancer development from initiation to metastasis through progression. In particular, as c-Met is known to be involved in drug resistance in the action mechanism of existing known anticancer drugs, the importance of individual customized treatment is further recognized, and c-Met has become a target molecule that many pharmaceutical companies are paying attention to in relation to anticancer drugs. Recently, many anti-cancer agents have been developed as antibodies against c-Met as an antagonist.

However, the effect of c-Met activation on proliferation and stem cell characteristics during subculture of stem cells is not well known.

### SUMMARY

As a result of analyzing various changes in characteristics of stem cells after adding c-Met agonist antibodies to a culture medium of stem cells, and then culturing stem cells and equally culturing stem cells in media to which a known c-Met ligand HGF was also added and/or not added, the present inventors confirmed that the c-Met agonist antibodies inhibited the extension of a population doubling time of stem cells, and the surface antigen expression and differentiation potential of stem cells was also maintained together with the effect of maintaining such proliferation ability, thereby completing the present disclosure.

Thus, an object of the present disclosure is to provide a medium additive for inhibiting the extension of a population doubling time (PDT) of stem cells, comprising an agonist antibody that specifically binds to c-Met as an active ingredient, and a medium composition for culturing stem cells, comprising the medium additive.

Further, another object of the present disclosure is to provide a method for inhibiting the extension of a population doubling time (PDT) of stem cells, the method comprising: culturing stem cells in the culture medium.

However, technical problems to be solved by the present disclosure are not limited to the aforementioned problems, and other problems that are not mentioned may be clearly understood by those skilled in the art from the following description.

In order to achieve the objects of the present disclosure as described above, the present disclosure provides a medium additive for inhibiting the extension of a population doubling time (PDT) of stem cells, comprising an agonist antibody that specifically binds to tyrosine-protein kinase Met (c-Met) as an active ingredient.

In addition, the present disclosure provides a medium composition for culturing stem cells, comprising the medium additive.

Furthermore, the present disclosure provides a method for inhibiting the extension of a population doubling time (PDT) of stem cells, the method comprising: culturing stem cells in the medium composition.

As an exemplary embodiment of the present disclosure, the stem cell may be an embryonic stem cell or an adult stem cell.

As another exemplary embodiment of the present disclosure, the adult stem cell may be a mesenchymal stem cell derived from one or more tissues selected from the group consisting of the umbilical cord, cord blood, bone marrow, fat, muscle, nerve, skin, the amnion, and the placenta.

As still another exemplary embodiment of the medium composition may inhibit the extension of a population doubling time extension of stem cells after passage 5.

As yet another exemplary embodiment of the present disclosure, the medium composition may maintain the differentiation potential of stem cells for 5 passages or more.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 illustrates the results of measuring population doubling times (PDTs), cell sizes, and cell viabilities while culturing mesenchymal stem cells up to passage 12 (P12) using a stem cell culture medium (#A58_ver2_Ab_3-1F6) to which an agonist antibody that specifically binds to the c-Met prepared in the present disclosure is added, a culture medium (#A58_ver2_HGF) to which HGF, which is a known ligand of c-Met, is added, and a culture medium (#A58_ver2_HGF free) to which HGF is not added;

FIGS. 2A, 2B, 2C, 2D, 2E, 2F and 2G illustrate the results of analyzing whether various surface antigens are expressed for mesenchymal stem cells cultured in a stem cell culture medium (Ver2_Ab_3-1F6) to which the c-Met agonist antibody is added, a culture medium (Ver2_HGF) to which HGF is added, and a culture medium (Ver2_HGF free) to which HGF is not added, respectively by FACS; and

FIGS. 3A, 3B, 3C and 3D illustrate the results of observing cell morphologies (FIG. 3A) and observing the degrees of differentiation after inducing differentiation into adipocytes (FIG. 3B), differentiation into chondrocytes (FIG. 3C), and differentiation into osteocytes (FIG. 3D) for mesenchymal stem cells cultured in stem cell culture media (A8 and A11) to which two types of c-Met agonist antibodies prepared in the present disclosure are respectively added, a culture medium (HGF(+)) to which HGF is added, and a culture medium to which HGF is not added, respectively.

### DETAILED DESCRIPTION

As a result of analyzing various changes in characteristics of stem cells after adding c-Met agonist antibodies to a culture medium of stem cells, and then culturing stem cells and equally culturing stem cells in media to which a known c-Met ligand HGF was also added and/or not added, the present inventors confirmed that the c-Met agonist antibodies inhibited the extension of a population doubling time of stem cells, and the surface antigen expression and differentiation potential of stem cells was also maintained together with the effect of maintaining such proliferation ability, thereby completing the present disclosure.

Thus, the present disclosure provides a medium additive for inhibiting extension of a population doubling time (PDT) of stem cells, comprising an agonist antibody that specifically binds to tyrosine-protein kinase Met (c-Met) as an active ingredient.

In addition, the present disclosure provides a medium composition for culturing stem cells, comprising the medium additive.

As used herein, the term agonist is also called an agent or an efficacy agent, and refers to a material exhibiting functions similar to those of a neurotransmitter, a hormone, and the like as a material that specifically binds to a receptor in vivo to change the higher-order structure thereof and imparts chemical information to cells. As used herein, the c-Met agonist antibody refers to an antibody capable of activating signaling through c-Met as with HGF known as a ligand of c-Met by specifically binding to c-Met, and specific sequences, morphologies, origins, synthesis methods, and the like are not limited as long as the c-Met agonist antibody is an antibody having such a function.

As used herein, the term “stem cell” refers to a cell having an ability to differentiate into two or more different cells, that is, stemness, while having a self-replication ability as an undifferentiated cell. The stem cell of the present disclosure may be an autologous or allogeneic-derived stem cell, and may be derived from any type of animal including a human and a non-human mammal, and is not limited to those derived from an adult body and derived from an embryo.

The stem cell of the present disclosure includes an embryonic stem cell or an adult stem cell, and is preferably an adult stem cell. The adult stem cell may be a mesenchymal stem cell, a human tissue-derived mesenchymal stromal cell, a human tissue-derived mesenchymal stem cell, a multipotent stem cell, or an amniotic epithelial cell, and is preferably a mesenchymal stem cell, but is not limited thereto. The mesenchymal stem cell may be a mesenchymal stem cell derived from the umbilical cord, cord blood, bone marrow, fat, muscle, nerve, skin, the amnion, the placenta, and the like, but is not limited thereto.

As used herein, the term “population doubling time (PDT) refers to a time until the number of cells as a whole population doubles by culturing. In general, the population doubling time refers to a time in the exponential growth phase before reaching saturation density and starting the inhibition of cell proliferation, and shows a constant amount according to the number of exponential growth phases. In normal human diploid cells, the population doubling time is elongated according to division aging, and is 16 hours in the early stages of culture and is prolonged to 40 hours or more in the late stages.

In the present disclosure, the inhibition of a population doubling time means that as stem cells are subcultured, the population doubling time in the early stages of culture is constantly maintained up to the late stages of culture by inhibiting the population doubling time from being prolonged as the culture proceeds unlike the initial stages of culture, and the c-Met agonist antibody of the present disclosure may inhibit the population doubling time of stem cells after passage 5 from being extended.

Furthermore, in the present disclosure, the medium composition may maintain the differentiation potential of stem cells for 5 passages or more.

As used herein, the term “differentiation potential” generally refers to an ability of a part of an embryo to differentiate into various organs or tissues under given developmental conditions in the early development of an organism, an ability to have all the differentiation abilities possible for the species is called pluripotency, an ability to differentiate into multiple types of cells is called multipotency, and an ability to differentiate into only one cell is called unipotency. Stem cells have a differentiation ability which is a unique ability that allows one cell to differentiate into a plurality of different types of cells, and the mesenchymal stem cells used in the present disclosure have multipotent characteristics.

In the present disclosure, it was confirmed through examples that an agonist antibody that specifically binds to c-Met is essential for maintaining the PDT of stem cells along with maintaining characteristics of stem cells, that is, the surface antigen expression and multipotency.

More specifically, in an exemplary embodiment of the present disclosure, as a result of measuring population doubling times, cell sizes, and cell viabilities while culturing mesenchymal stem cells up to passage 12 (P12) using a stem cell culture medium to which the c-Met agonist antibody prepared in the present disclosure was added, a culture medium to which HGF was added, and a culture medium to which neither the antibody nor HGF was added, respectively, it was confirmed that in the case of the stem cells cultured in the stem cell culture medium to which the c-Met agonist antibody was added, the PDT was constantly maintained without being increased up to passage 12 unlike stem cells cultured in other media (see Example 3).

In another exemplary embodiment of the present disclosure, the surface antigen expression and differentiation potential of stem cells was analyzed in order to see whether other characteristics of stem cells were also maintained in addition to an effect in which the PDT of stem cells was maintained by adding the c-Met agonist antibody. As a result, it was confirmed that in the case of stem cells cultured in the medium to which the c-Met agonist antibody was added, the surface antigen was expressed at a higher level at passage 7 unlike the stem cells cultured in a control medium to which neither the antibody nor HGF was added (see Example 4-1), and as a result of culturing cells up to passage 10 and inducing differentiation into adipocytes, chondrocytes, and osteocytes, through the fact that the stem cells were well differentiated into adipocytes, chondrocytes, and osteocytes, respectively, it was confirmed that the multipotency was well maintained (see Example 4-2).

Through the example results, it was confirmed that the culture composition including the c-Met agonist antibody according to the present disclosure has effects of maintaining the proliferation potential, in other words, the self renewal ability, of stem cells and also inhibiting the differentiation potential from being reduced by inhibiting a population doubling time from being extended while maintaining the undifferentiation state of stem cells for 5 passages or more, so that overall, the results of exemplary embodiments of the present disclosure demonstrate that the c-Met agonist antibody is very important for maintaining the stemness of stem cells during subculture.

Thus, as another aspect of the present disclosure, the present disclosure provides a method for maintaining a population doubling time (PDT) of stem cells, the method comprising: culturing stem cells in the medium composition.

Hereinafter, preferred examples for helping the understanding of the present disclosure will be suggested. However, the following examples are provided only to more easily understand the present disclosure, and the contents of the present disclosure are not limited by the following examples.

### EXAMPLES

### Example 1. Cell Culture

The mesenchymal stem cell used in the examples of the present disclosure is an adipose-derived mesenchymal stem cell, and the cells were cultured using a culture medium purchased from Xcell Therapeutics Inc.

### Example 2. Preparation of c-Met Agonist Antibody

2-1. Phage Display

A human recombinant c-Met protein was used as an antigen, and a human ScFv library was used for screening for hits that bind to a c-Met extracellular region. The antigen was coated on an immunotube (Nunc, USA) at a concentration of 10 μg/μl and cultured with O/N and bound. Then, activities of the immunotube and a phage were inhibited with a blocking buffer (3% milk in PBST). Next, the phage was put into the antigen-coated immunotube and bound, and after 1 hour, washed four times with PBST and one time with PBS, and the phage was eluted in 100 mM TEA for 7 to 8 minutes and then neutralized with a Tris-HCl (pH 8) solution. E. coli was infected with the eluted phage, and an output titer was confirmed by culturing some with O/N in a solid LA plate. The remaining phage was rescued using a helper phage, and the same experiment was repeated three times.

2-2. ELISA Screening

After the fourth panning, a single colony was injected into 150 μl of SB containing ampicillin in each 96-well plate, and cultured in a 37° C. shake incubator until the medium became cloudy. After cultivation, the culture solution was put into the original plate, induced with 1 nM isopropyl β-D-1-thiogalactopyranoside (IPTG), and then cultured at 30° C. overnight. A c-Met recombinant protein was used as an antigen, dissolved in PBS at a concentration of 1 μg/ml on an ELISA plate (Corning 3690) for coating, and cultured at 4° C. overnight. The next day, after the clone-injected plate was centrifuged at 3000 rpm for 15 minutes, the supernatant was removed, the pellet was resuspended at 37° C. in a 1×TES buffer for 5 to 7 minutes, and then a 0.2×TES buffer was added thereto to react at 4° C. for 30 minutes, thereby lysing the cells. An antigen-coated plate was washed three times with 150 μl TBST and the reaction was inhibited using 3% skim milk. Then, a periplasmic extract was obtained from the lysed cells, and the reaction was inhibited using 6% skim milk in a new plate for 1 hour. The solution was then added to the antigen-coated plate, cultured at room temperature for 1 hour at constant temperature, and then washed three times using TBST. Then, an anti-HA Hrp secondary antibody was added thereto, the resulting mixture was cultured for 1 hour, and then washed three times with TBST. Then, the reaction was started by performing treatment with 30 μl of TMB, and then the reaction was inhibited using 1N H2504, and absorbance was detected at 450 nm.

2-3. IgG Conversion, Overexpression, and Antibody Purification

An attempt was made to convert the final hits selected after the ELISA screening into human IgG. Based on the ELISA results, each candidate was named A8, A9, A11, B8, B10, C8, C9, D7, D12, and E10, and 10 hits were converted to human IgG forms. More specifically, the scFv sequence was converted to human light and heavy chain sequences and fused to pOptiVEC™-TOPO and pcDNA™3.3-TOPO (Thermo Fisher, USA) vectors by cloning. The plasmid was then amplified using Midi prep (Macherey Nagel, Germany).

The amplified plasmid was temporarily expressed using a FreeStyle Expression System (Invitrogen, USA). FreeStyle cells were thawed in a FreeStyle Expression Medium using an Erlenmeyer flask (Corning, USA) and cultured. Cells were cultured until the concentration of 3.0×10⁶ cells/ml and subcultured every 2 and 3 days, and after 4 passages, heavy and light chain plasm ids were transformed using FreeStyle™ MAX Transfection Reagent (Invitrogen, USA), and cells were cultured in a shaker under 8% CO₂, 37° C. conditions. Cells were obtained on day 7 after culture, a supernatant was obtained and filtered, and then the supernatant was applied to MabSelect SuRe protein A beads (GE Healthcare. USA) in a chromatography column (Bio-Rad, USA). The size was then confirmed by SDS-PAGE and Coomassie blue staining.

2-4. Final Selection of Antibody

4 types of antibodies (A8, A11, B10, and C8) having specificity for c-Met were selected by confirming the presence or absence of binding to natural c-Met using the 10 types of hit candidate antibodies. Then, the binding affinity for c-Met was analyzed, and A8 and A11 antibodies were finally selected by verifying whether signal activity was induced by binding to c-Met like HGF.

### Example 3. Confirmation of Stem Cell PDT Maintenance Effect by c-Met Agonist Antibody

To examine the effect of the c-Met agonist antibody prepared in Example 2 on a long-term culture of stem cells, the present inventors measured the population doubling time (PDT), cell size, and cell viability while culturing stem cells up to passage 12 (P12) in a stem cell culture medium (#A58_ver2_Ab_3-1F6) to which one of the prepared antibodies was added. In this case, stem cells were cultured under the same conditions using, as controls, a medium (#A58_ver2_HGF) to a hepatocyte growth factor (HGF) known as a c-Met ligand was added and a stem cell culture medium (#A58_ver2_HGF free) which is the same as the previous stem cell culture medium except that HGF was not added, respectively.

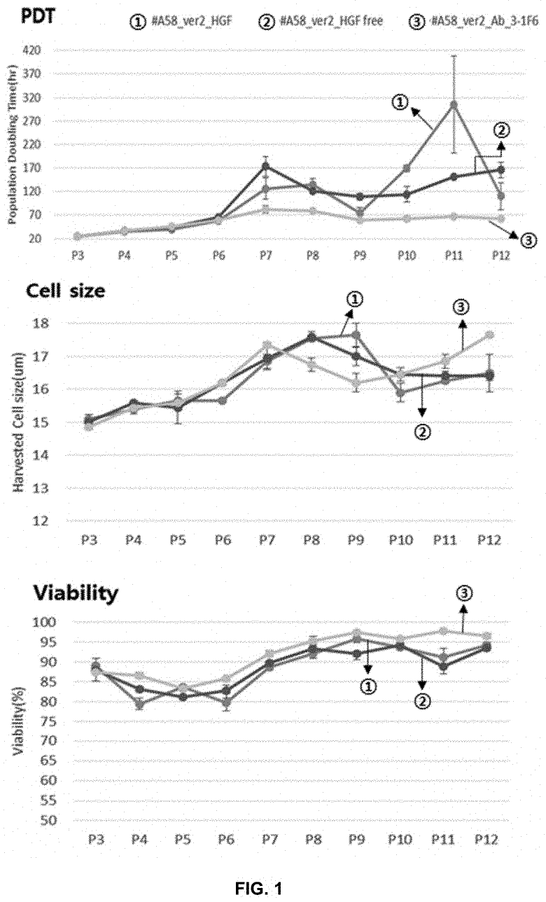

As a result of analyzing the PDT, size and viability of the cells at each passage while culturing the cells up to passage 12, as illustrated in FIG. 1, there is no significant difference in cell size and viability between the stem cells cultured in each medium, but in the case of PDT, it was confirmed that the stem cells cultured in the medium to which the c-Met agonist antibody was added maintained an almost constant PDT of around 70 hours up to passage 12. In contrast, it was shown that in the stem cells cultured in the medium to which HGF was added and the medium to which HGF was not added, the PDT began to show a slight difference from passage 5, was noticeably increased from passage 6, and was not constantly maintained. Through the results, it could be seen that the c-Met agonist antibody according to the present disclosure had effects of maintaining the size and viability of stem cells up to passage 12 and also maintaining a constant PDT, and these effects are specific effects by the antibody, which are not shown by adding the HGF which is a c-Met ligand.

### Example 4. Confirmation of Stem Cell Characteristic Maintenance Effect by c-Met Agonist Antibody

In addition to the result of Example 3, the present inventors intended to verify, when stem cells were subcultured in the medium to which the c-Met agonist antibody was added, whether inherent characteristics of stem cells were maintained by the c-Met agonist antibody during subculture of the stem cells.

4-1. Surface Antigen Expression Analysis of Stem Cells

First, in order to analyze the expression degree of the surface antigen of stem cells, it was analyzed whether an antigen expressed on the cell surface was expressed by FACS using cells of passage 7 and passage 12 while culturing the mesenchymal stem cells of Example 1 up to passage 12 using the culture medium to which the c-Met agonist antibody was added, the medium to which HGF was added, and the medium to which HGF was not added, respectively.

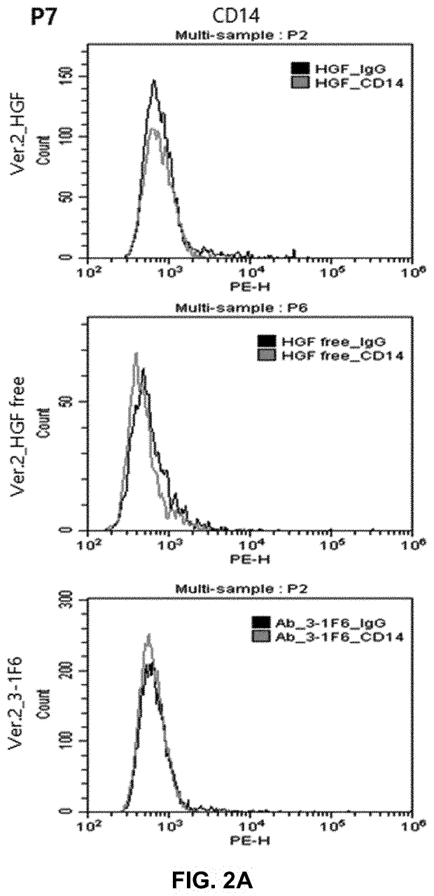

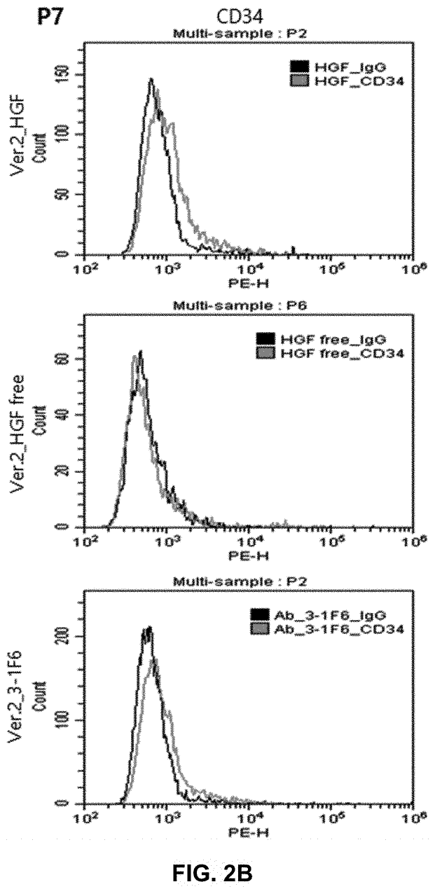

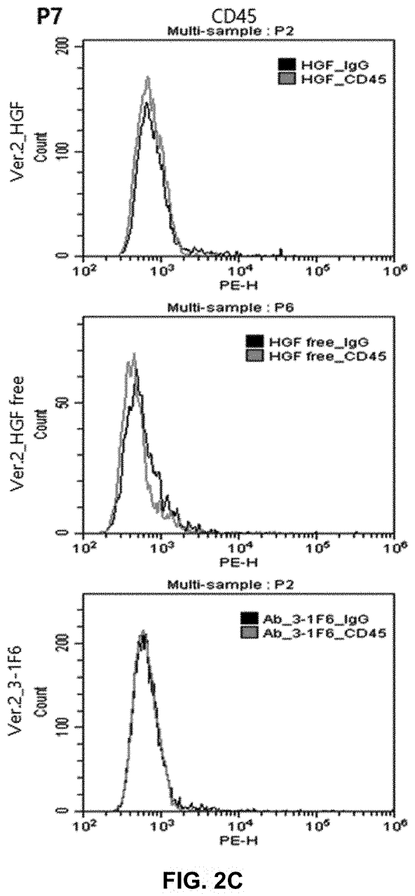

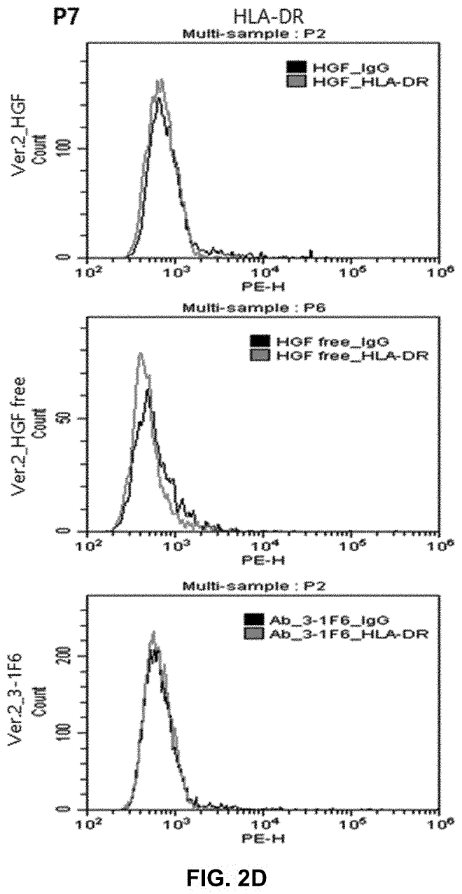

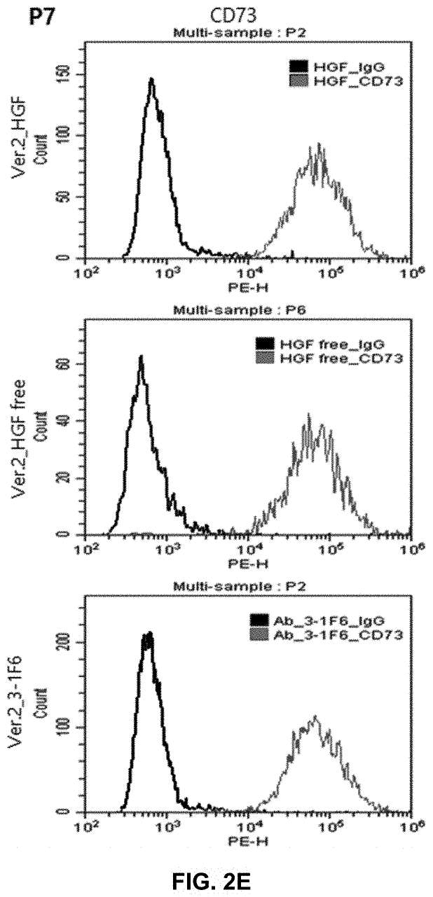

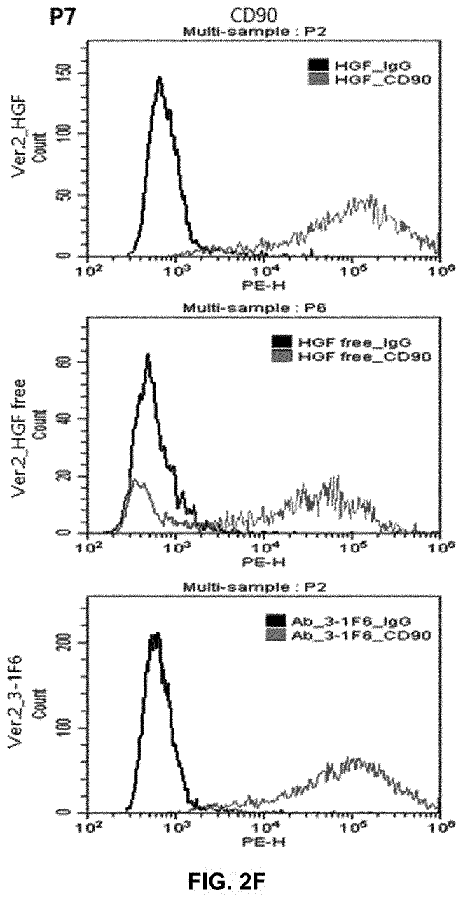

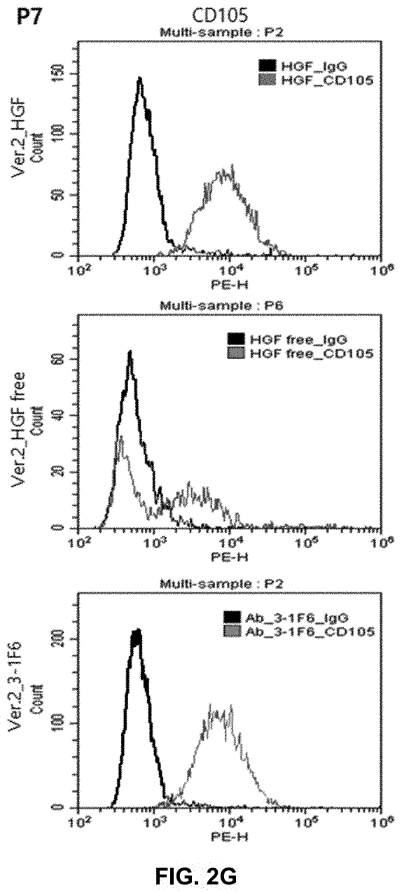

As a result, as illustrated in FIG. 2A to 2G, it was confirmed that in the case of cells of passage 7, both the stem cells cultured in the medium to which the c-Met agonist antibody or HGF was added and the medium to which HGF was not added did not express blood cell-specific antigens CD14, CD34, CD45, and HLA-DR. Further, it was shown that in the case of CD73, CD90 and CD105, which are known to be expressed in mesenchymal stem cells, the stem cells cultured in the medium to which the c-Met agonist antibody was added expressed the antigens at higher levels than the stem cells cultured in the medium to which HGF was not added. In addition, it was confirmed that such an expression pattern appeared similar to that of stem cells cultured in the medium to which HGF was added, and was maintained up to passage 12. Through the results, it could be seen that as stem cells were subcultured, a pattern in which the expression of the surface antigen was reduced could appear, but the expression of the surface antigen was maintained until the late stage by adding the c-Met agonist antibody.

4-2. Differentiation Potential Maintenance Analysis of Stem Cells

Furthermore, the present inventors intended to verify whether the differentiation potential of stem cells was maintained by the c-Met agonist antibody during subculture of stem cells. For this purpose, the two types of c-Met-specific agonist antibodies finally selected in Example 2 were added to the culture medium of stem cells and cultured, and then the differentiation potential of stem cells was analyzed.

More specifically, the adipose-derived mesenchymal stem cells of Example 1 was cultured up to passage 9 (P9) in a medium in which the c-Met specific agonist antibody was added (A8 or A11) or HGF was added (HGF(+)) or HGF was not added (HGF(−)) to a stem cell culture medium having the same composition. Then, the mesenchymal stem cells of passage 9 were inoculated into a 6-well plate and then grown according to the cell lineage to differentiate, and specifically induced to differentiate into fat, cartilage, and bone.

In order to differentiate the mesenchymal stem cells into adipocytes, the culture medium was replaced with a cell differentiation medium when the cells grew to about 90 to 100% at passage 10. A StemPro adipogenesis differentiation kit (Thermo Fisher, USA) was used as a differentiation medium, and the medium was changed every 2 to 3 days, and maintained for 2 to 3 weeks. After differentiation into fat, the cells were stained with Oil red 0 for observation.

Furthermore, in order to differentiate the mesenchymal stem cells into chondrocytes, the culture medium was replaced with a cell differentiation medium when the cells grew to about 50% at passage 10. A DMEM low-glucose medium (Welgene, Korea) containing FBS (Hyclone, USA), 1% ITS-X, 50 μg/ml ascorbic acid, 100 nM dexamethasone, and 10 ng/ml TGF-β1 was used as a differentiation medium, and the medium was changed every 2 to 3 days, and maintained for 2 to 3 weeks. After 3 weeks, the cells were fixed, stained with Alcian Blue and observed.

Further, in order to differentiate the mesenchymal stem cells into osteocytes, the cells were cultured up to passage 10, and then the culture medium was replaced with a cell differentiation medium. A DMEM low-glucose medium (Welgene, Korea) containing FBS, 100 nM dexamethasone, 10 nM glycerol-2-phosphate, 50 ug/ml ascorbic acid, and 1% GlutaMAX was used as a differentiation medium, and the medium was changed every to 3 days, and maintained for 3 weeks. After 3 weeks, the cells were fixed, stained with an Alizarin Red solution, and observed.

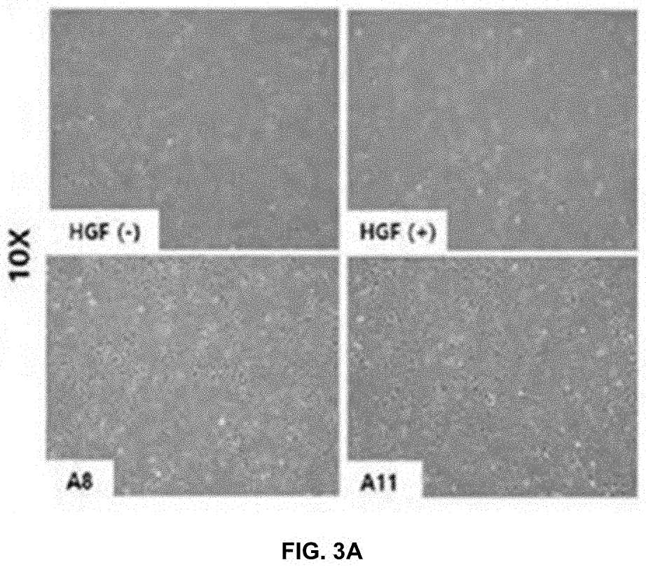

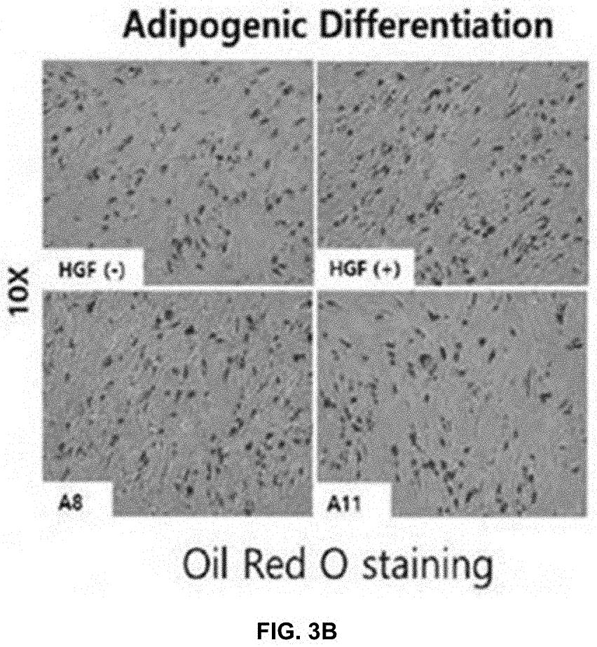

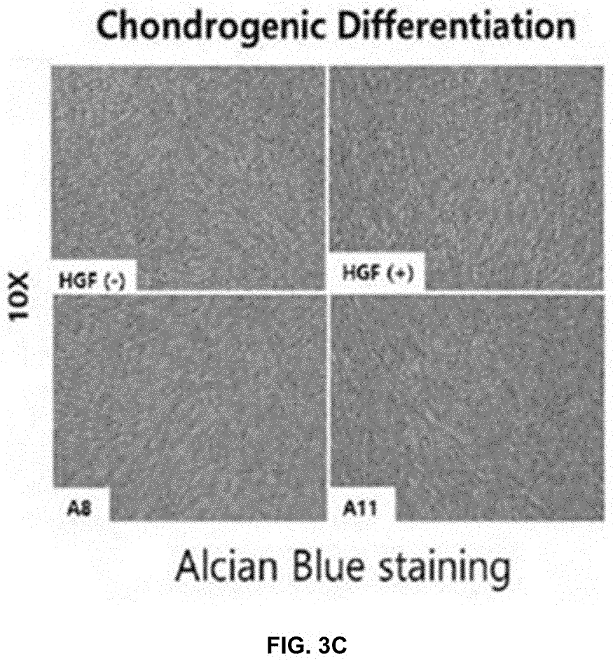

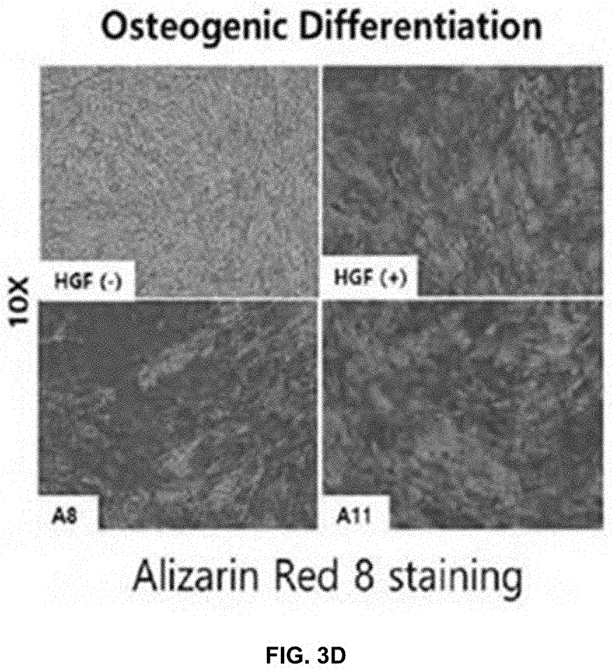

Overall, the degree of cell proliferation and differentiation was observed in each case of mesenchymal stem cells cultured in a medium without HGF and mesenchymal stem cells cultured in a medium containing HGF or the c-Met agonist antibody (A8 or A11). As a result, as illustrated in FIGS. 3A to 3D, stem cells were found to proliferate at a similar level (FIG. 3A) and differentiate into adipocytes under all media conditions. However, it was confirmed that in the case of stem cells cultured in a medium without HGF, differentiation into chondrocytes and osteocytes did not occur, and unlike this case, in the case of stem cells cultured in a medium to which the c-Met agonist antibody was added, differentiation into all cells, which were induced to differentiate, occurred.

Through this, it could be seen that stem cells cultured in the medium to which the c-Met agonist antibody was added were effectively maintained without a reduction in differentiation potential, and it could be confirmed that the c-Met agonist antibody played an important role in maintaining the differentiation potential.

Through specific experiments, the present inventors confirmed that when stem cells were subcultured by adding a c-Met agonist antibody to a medium, the extension of a population doubling time (PDT) of stem cells was effectively inhibited to constantly maintain the PDT, and the surface antigen expression and differentiation potential of mesenchymal stem cells is also maintained after passage 5, compared to media to which HGF was added or not added, and since the proliferation and differentiation potential of stem cells can be continuously maintained when the stem cells were cultured in a medium composition to which a c-Met agonist antibody was added, the c-Met agonist antibody according to the present disclosure is expected to be used as a simple and efficient means which can inhibit a reduction in proliferation and differentiation potential of stem cells by the subculture of stem cells and effectively maintain the ability in stem cell studies and the field of development of a therapeutic agent using the c-Met agonist antibody according to the present disclosure.

The above-described description of the present disclosure is provided for illustrative purposes, and those skilled in the art to which the present disclosure pertains will understand that the present disclosure can be easily modified into other specific forms without changing the technical spirit or essential features of the present disclosure. Therefore, it should be understood that the above-described embodiments are only exemplary in all aspects and are not restrictive.

What is claimed is:

1. A medium additive for inhibiting the extension of a population doubling time (PDT) of stem cells, comprising an agonist antibody that specifically binds to tyrosine-protein kinase Met (c-Met) as an active ingredient.

2. A medium composition for culturing stem cells, comprising the medium additive of claim 1.

3. The medium composition of claim 2, wherein the stem cell is an embryonic stem cell or an adult stem cell.

4. The medium composition of claim 3, wherein the adult stem cell is a mesenchymal stem cell derived from one or more tissues selected from the group consisting of the umbilical cord, cord blood, bone marrow, fat, muscle, nerve, skin, the amnion, and the placenta.

5. The medium composition of claim 2, wherein the medium composition inhibits the extension of a population doubling time of stem cells after passage 5.

6. The medium composition of claim 2, wherein the medium composition maintains the differentiation potential of stem cells for 5 passages or more.

7. A method for inhibiting the extension of a population doubling time (PDT) of stem cells, the method comprising:
culturing stem cells in the medium composition of claim 2.

8. The method of claim 7, wherein the stem cell is an embryonic stem cell or an adult stem cell.

9. The method of claim 8, wherein the adult stem cell is a mesenchymal stem cell derived from one or more tissues selected from the group consisting of the umbilical cord, cord blood, bone marrow, fat, muscle, nerve, skin, the amnion, and the placenta.
